(19) United States
(12) Patent Application Publication
(10) Pub. No.: US 2025/0152610 A1
(43) Pub. Date: May 15, 2025

(54) IMIDAZOLATE GOLD COMPOUNDS FOR THE TREATMENT OF LUNG CANCER

(71) Applicant: University of Cincinnati, Cincinnati, OH (US)

(72) Inventors: Cristina Andreani, Cincinnati, OH (US); Caterina Bartolacci, Cincinnati, OH (US); Pier Paolo Scaglioni, Cincinnati, OH (US); Rossana Galassi, Camerino (IT); Stefania Pucciarelli, Camerino (IT)

(73) Assignee: University of Cincinnati, Cincinnati, OH (US)

(21) Appl. No.: 18/841,158

(22) PCT Filed: Feb. 24, 2023
(86) PCT No.: PCT/US2023/013848

§ 371 Date: Aug. 23, 2024

Related U.S. Application Data
(60) Provisional application No. 63/314,029, filed on Feb. 25, 2022.

(51) Int. Cl. A61K 31/675; A61K 45/06; A61P 35/00

(52) U.S. Cl. CPC A61K 31/675; A61K 45/06; A61P 35/00

(57) ABSTRACT

Methods of treating lung cancer in a subject in need thereof are provided, including administering to the subject an effective amount of an imidazolate gold compound according to Formula I, or a pharmaceutically acceptable salt, racemate, or enantiomer thereof:

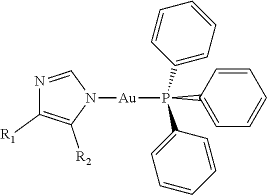

wherein R₁ and R₂ are independently selected from the group consisting of halo, cyano, hydroxyl, carboxyl, alkyl, aryl, esteryl, amidyl, and alkoxyl. Methods of inducing ferroptosis in a lung cancer cell and pharmaceutical compositions are also provided.

### CROSS REFERENCE TO RELATED APPLICATION

This application claims priority to U.S. Provisional Application Ser. No. 63/314,029, filed Feb. 25, 2022, the entire contents of which are incorporated herein by reference.

### TECHNICAL FIELD

This disclosure relates to methods of treating lung cancer by administering imidazolate gold compounds.

### BACKGROUND

Approximately 80-85% of lung cancers are non-small cell lung cancer (NSCLC). The most common subtypes of NSCLC include adenocarcinoma, squamous cell carcinoma, and large cell carcinoma, while less common subtypes include adenosquamous carcinoma and sarcomatoid carcinoma. Although NSCLC typically progresses more slowly than small cell lung cancer (SCLC), 40% of NSCLCs have metastasized beyond the lungs by the time the cancer is diagnosed.

The presence or absence of mutations in the Kirsten rat sarcoma viral oncogene (KRAS) has been identified as a factor affecting the responsiveness of NSCLC to anti-cancer therapies. Early diagnosis and accurate characterization of lung cancer improves the likelihood of a positive prognosis for NSCLC.

A need exists for targeted therapies for treating NSCLC.

### SUMMARY

Accordingly, the present disclosure is directed to targeted therapies for lung cancer, namely, imidazolate gold compounds that induce ferroptosis in lung cancer cells, thereby inducing programmed cell death.

In one embodiment, a method of treating lung cancer in a subject in need thereof is provided, the method comprising administering to the subject an effective amount of an imidazolate gold compound according to Formula I, or a pharmaceutically acceptable salt, racemate, or enantiomer thereof:

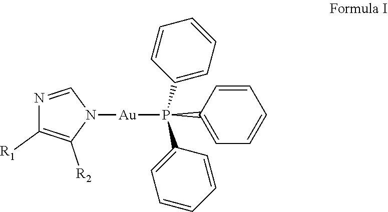

wherein R₁ and R₂ are independently selected from the group consisting of halo, cyano, hydroxyl, carboxyl, alkyl, aryl, amidyl, alkoxyl, and —C(O)—OR₃, wherein R₃ is a C1-C12 alkyl.

In another embodiment, a method of inducing ferroptosis in a lung cancer cell is provided, the method comprising contacting the lung cancer cell with an effective amount of a Formula I compound or a pharmaceutically acceptable salt, racemate, or enantiomer thereof.

In another embodiment, a compound according to Formula I, or a pharmaceutically acceptable salt, racemate, or enantiomer thereof, for use in treating lung cancer is provided.

In another embodiment, a pharmaceutical composition for the treatment of lung cancer is provided, the composition comprising an effective amount of a Formula I compound or a pharmaceutically acceptable salt, racemate, or enantiomer thereof; and at least one pharmaceutically acceptable excipient.

These and other objects, features, embodiments, and advantages will become apparent to those of ordinary skill in the art from a reading of the following detailed description and the appended claims.

### BRIEF DESCRIPTION OF THE DRAWINGS

The details of embodiments of the presently-disclosed subject matter are set forth in this document. Modifications to embodiments described in this document, and other embodiments, will be evident to those of ordinary skill in the art after a study of the information provided herein.

FIG. 1 is a representation of the cellular pathways that regulate ferroptosis. Adapted from Zheng J, Conrad M., The Metabolic Underpinnings of Ferroptosis, Cell Metabolism 32(6): 920-37 (2020).

FIG. 2A is a graph showing CERES scores from DepMap for thioredoxin reductase (TXNRD1) in wild type KRAS (KRAS-WT) and mutant KRAS (KM) human lung cancer cells.

FIG. 2B is a graph showing CERES scores from DepMap for glutathione peroxidase 4 (GPX4) in KRAS-WT and KM human lung cancer cells.

FIG. 2C is a graph showing CERES scores from DepMap for apoptosis-inducing factor 2 (AIFM2) in KRAS-WT and KM human lung cancer cells.

FIG. 2D is a graph showing CERES scores from DepMap for coenzyme Q10A (COQ10A) in KRAS-WT and KM human lung cancer cells.

FIG. 2E is a graph showing CERES scores from DepMap for GTP cyclohydrolase 1 (GCH1) in KRAS-WT and KM human lung cancer cells.

FIG. 2F is a graph showing CERES scores from DepMap for DHFR in KRAS-WT and KM human lung cancer cells.

FIG. 3A is a graph depicting MTT viability assay results for KRAS-WT, KM, and IMR₉₀ lung fibroblasts treated with auranofin.

FIG. 3B is a graph showing IC₅₀ values for KRAS-WT and KM cells treated with auranofin.

FIG. 3C is a graph depicting MTT viability assay results for KRAS-WT, KM, and IMR₉₀ lung fibroblasts treated with DM20.

FIG. 3D is a graph showing IC₅₀ values for KRAS-WT and KM cells treated with DM20.

FIG. 3E is a graph depicting MTT viability assay results for KRAS-WT, KM, and IMR₉₀ lung fibroblasts treated with CS47.

FIG. 3F is a graph showing IC₅₀ values for KRAS-WT and KM cells treated with CS47.

FIG. 4A depicts MTT cell viability assay results for H522 KRAS-WT lung cancer cells after 48 hr treatment (in quadruplicate) with DM20+Ferrostatin-1 (left panel); CS47+Ferrostatin-1 (middle panel); and auranofin+Ferrostatin-1 (right panel).

FIG. 4B depicts MTT cell viability assay results for H522 KRAS-WT lung cancer cells after 48 hr treatment (in quadruplicate) with DM20+L-cysteine (left panel); CS47+L-cysteine (middle panel); and auranofin+L-cysteine (right panel).

FIG. 4C depicts MTT cell viability assay results for H522 KRAS-WT lung cancer cells after 48 hr treatment (in quadruplicate) with DM20+NAC (left panel); CS47+NAC (middle panel); and auranofin+NAC (right panel).

FIG. 5A is an image of a Western blot analysis for TXNRD1 and GPX4 on total protein lysates extracted from KM and KRAS-WT cells after 48 hrs treatment with the noted compounds. Results show treatment depletes GPX4 and induces lipid peroxidation in KRAS-WT cells.

FIG. 5B shows C11-BODIPY staining in KM and KRAS-WT cells after 48 hr treatment with the noted compounds. Results show treatment depletes GPX4 and induces lipid peroxidation in KRAS-WT cells.

### DETAILED DESCRIPTION

The details of one or more embodiments of the presently-disclosed subject matter are set forth in this document. Modifications to embodiments described in this document, and other embodiments, will be evident to those of ordinary skill in the art after a study of the information provided in this document.

While the following terms are believed to be well understood in the art, definitions are set forth to facilitate explanation of the presently-disclosed subject matter. Unless defined otherwise, all technical and scientific terms used herein have the same meaning as commonly understood by one of ordinary skill in the art to which the presently-disclosed subject matter belongs.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to achieve the disclosed subject matter.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

For the purposes of defining the present technology, the transitional phrase “consisting of” may be introduced in the claims as a closed preamble term limiting the scope of the claims to the recited components or steps and any naturally occurring impurities. For the purposes of defining the present technology, the transitional phrase “consisting essentially of” may be introduced in the claims to limit the scope of one or more claims to the recited elements, components, materials, or method steps as well as any non-recited elements, components, materials, or method steps that do not materially affect the novel characteristics of the claimed subject matter. The transitional phrases “consisting of” and “consisting essentially of” may be interpreted to be subsets of the open-ended transitional phrases, such as “comprising” and “including,” such that any use of an open ended phrase to introduce a recitation of a series of elements, components, materials, or steps should be interpreted to also disclose recitation of the series of elements, components, materials, or steps using the closed terms “consisting of” and “consisting essentially of.” For example, the recitation of a composition “comprising” components A, B, and C should be interpreted as also disclosing a composition “consisting of” components A, B, and C as well as a composition “consisting essentially of” components A, B, and C. Any quantitative value expressed in the present application may be considered to include open-ended embodiments consistent with the transitional phrases “comprising” or “including” as well as closed or partially closed embodiments consistent with the transitional phrases “consisting of” and “consisting essentially of.”

As used herein the singular forms “a,” “an” and “the” include plural references unless the context clearly indicates otherwise. The verb “comprises” and its conjugated forms should be interpreted as referring to elements, components or steps in a non-exclusive manner. The referenced elements, components or steps may be present, utilized or combined with other elements, components or steps not expressly referenced.

When the term “independently selected” is used, the substituents being referred to (e.g., R groups, such as groups R₁ and R₂), can be identical or different. For example, both R₁ and R₂ can be the same substituent, or R₁ and R₂ can each be different substituents selected from a specified group.

It should be understood that any two quantitative values assigned to a property may constitute a range of that property, and all combinations of ranges formed from all stated quantitative values of a given property are contemplated in this disclosure.

The term “subject,” as used herein, means any mammalian subject, including humans.

The terms “treat,” “treatment,” and “treating,” as used herein, refer to a method of alleviating or abrogating a disease, disorder, and/or symptoms thereof.

An “effective amount,” as used herein, refers to an amount of a substance (e.g., a therapeutic compound and/or composition) that elicits a desired biological response. In some embodiments, an effective amount of a substance is an amount that is sufficient, when administered to a subject suffering from or susceptible to a disease, disorder, and/or condition, to treat, diagnose, prevent, and/or delay and/or alleviate one or more symptoms of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of a formulation to treat a disease, disorder, and/or condition is the amount that alleviates, ameliorates, relieves, inhibits, prevents, delays onset of; reduces severity of and/or reduces incidence of one or more symptoms or features of the disease, disorder, and/or condition. Furthermore, an effective amount may be administered via a single dose or via multiple doses within a treatment regimen. In some embodiments, individual doses or compositions are considered to contain an effective amount when they contain an amount effective as a dose in the context of a treatment regimen. Those of ordinary skill in the art will appreciate that a dose or amount may be considered to be effective if it is or has been demonstrated to show statistically significant effectiveness when administered to a population of patients; a particular result need not be achieved in a particular individual patient in order for an amount to be considered to be effective as described herein.

The terms “halo” or “halogen,” as used herein, refer to fluoro (F), chloro (CI), bromo (Br), and iodo (I) groups.

The terms “cyano” or “nitrile,” as used herein, refer to a —C≡N functional group.

The term “hydroxyl,” as used herein, refers to a —OH functional group.

The term “carboxyl,” as used herein, refers to a —COOH functional group.

The term “alkyl,” as used herein, refers to a straight or branched saturated aliphatic hydrocarbon group having a single radical and 1-12 carbon atoms (i.e., C1-C12 alkyl). Non-limiting examples of alkyl groups include methyl, propyl, isopropyl, butyl, n-butyl, isobutyl, sec-butyl, tert-butyl, pentyl, hexyl, heptyl, octyl, nonyl, and the like. A branched alkyl means that one or more alkyl groups such as methyl, ethyl, or propyl replace one or both hydrogens in a —CH₂— group of a linear alkyl chain. In certain embodiments, alkyl is a C1-C6 alkyl or a C1-C4 alkyl. In other embodiments, alkyl is selected from the group consisting of methyl, ethyl, propyl, isopropyl, butyl, n-butyl, isobutyl, sec-butyl, and tert-butyl.

Alkyl groups can optionally be unsubstituted or substituted (a “substituted alkyl”) with one or more alkyl group substituents, which can be the same or different. The term “alkyl group substituent” includes but is not limited to alkyl, substituted alkyl, halo, hydroxyl, carboxyl, oxo, and the like. There can be optionally inserted along the alkyl chain one or more oxygen, sulfur or substituted or unsubstituted nitrogen atoms, wherein the nitrogen substituent is hydrogen or alkyl.

The term “oxo,” as used herein, refers to a ═O functional group.

The term “aryl,” as used herein, refers to an optionally substituted polyunsaturated aromatic group. The aryl may contain a single ring (i.e., phenyl) or more than one ring, wherein at least one ring is aromatic. When the aryl comprises more than one more ring, the rings may be fused, linked via a covalent bond (for example biphenyl). The aromatic ring may optionally comprise one to two additional fused rings (i.e., cycloalkyl, heterocycloalkyl, or heteroaryl). Examples include phenyl, naphthyl, biphenyl, phenanthrenyl, naphthacenyl, and the like. In specific embodiments, the aryl is substituted or unsubstituted phenyl.

The term “esteryl,” as used herein, refers to a —C(O)—OR₃ functional group, wherein R₃ may be an aryl or a C1-C12 alkyl. In specific embodiments, R₃ is methyl, ethyl, or propyl.

The term “amidyl,” as used herein, refers to a —C(O)NR₄ functional group, wherein R₄ is hydrogen, alkyl, or aryl as previously described. In embodiments, amidyl includes primary, secondary, or tertiary amines, such as —C(O)NH₂, —C(O)NHR₄, and —C(O)N(R₄)₂ where R₄ may be an aryl or a C1-C12 alkyl.

The term “alkoxyl,” as used herein, refers to an-O-alkyl functional group wherein alkyl is as previously described. The term “alkoxyl” as used herein can refer to, for example, one or more of methoxyl, ethoxyl, propoxyl, isopropoxyl, butoxyl, t-butoxyl, pentoxyl, and the like. In certain embodiments, alkoxyl is a C1-C12 alkoxyl, a C1-C6 alkoxyl, or a C1-C4 alkoxyl.

A “pharmaceutically acceptable salt” is a cationic salt formed at any acidic (e.g., hydroxamic or carboxylic acid) group, or an anionic salt formed at any basic (e.g., amino) group. Many such salts are known in the art, as described in WO 1987/005297, by Johnston et al., published Sep. 11, 1987. Specific cationic salts include the alkali metal salts (such as sodium and potassium), and alkaline earth metal salts (such as magnesium and calcium) and organic salts. Specific anionic salts include halide (such as chloride, bromide, or fluoride salts), sulfate, and maleate. In embodiments, suitable pharmaceutically acceptable salts include, but are not limited to, halide, sodium, sulfate, acetate, phosphate, diphosphate, potassium, maleate, calcium, citrate, mesylate, nitrate, tartrate, aluminum, gluconate, carboxylate, and the like.

Such salts are well understood by the skilled artisan and the skilled artisan is able to prepare any number of salts given the knowledge in the art. Furthermore, it is recognized that the skilled artisan may select one salt over another for reasons of solubility, stability, formulation ease and the like. Determination and optimization of such salts is within the purview of the skilled artisan's practice.

The terms “enantiomer” and “racemate” have the standard art recognized meanings (see, e.g., Hawley's Condensed Chemical Dictionary, 16th ed. (2016)). The illustration of specific protected forms and other derivatives of the compounds of the instant invention is not intended to be limiting. The application of other useful protecting groups, salt forms, esters, and the like is within the purview of the skilled artisan.

Ferroptosis is an iron-dependent, non-apoptotic form of programmed cell death triggered by abnormal metabolic and biochemical processes that exert acute or chronic stress on a cell. Ferroptosis has been associated with diseases including acute kidney injury, cardiovascular, neurodegenerative disease, hepatic disease, and cancer. Ferroptosis has been identified as a promising target for developing new cancer therapies.

Mutant KRAS lung cancer (KMLC) is dependent on fatty acid synthesis and metabolism. Upon inhibition of fatty acid synthase (FASNi), the rate-limiting enzyme of fatty acid synthesis, KMLC cells undergo ferroptosis both in vitro and in vivo in preclinical models. However, lung cancer harboring no KRAS mutations (KRAS-WT) are resistant to FASNi and to FASNi-induced ferroptosis.

Ferroptosis is characterized by lipid peroxidation. The process is defined by three primary hallmarks: the presence of oxidable phospholipids, the presence of redox-active iron that catalyzes the oxidation process, and an impaired or defective lipid peroxide repair system.

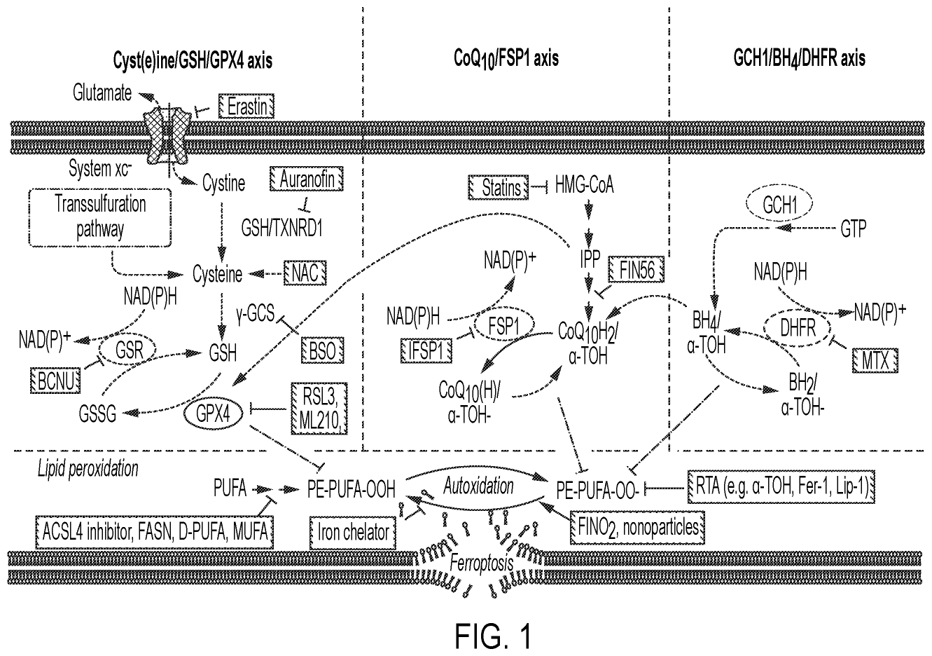

Ferroptosis is suppressed via three main pathways that regulate lipid peroxidation, as depicted in FIG. 1: (1) the cyst(e)ine/thioredoxin reductase/glutathione/glutathione peroxidase 4 (cyst(e)ine/TXNRD1/GSH/GPX4) axis; (2) the coenzyme Q10/ferroptosis suppressor protein 1 (CoQ10/FSP1) axis; and (3) the GTP cyclohydrolase 1/tetrahydrobiopterin/dihydrofolate reductase (GCH1/BH4/DHFR) system. As illustrated in FIG. 1, lipid peroxidation is efficiently counteracted by the cyst (e) ine/TXNRD1/GSH/GPX4, CoQ10/FSP1, and GCH1/BH4/DHFR systems.

### Imidazolate Gold Compounds

The present disclosure relates to imidazolate gold compounds that induce ferroptosis in lung cancer cells, particularly KRAS-WT lung cancer cells. In embodiments, imidazolate gold compounds, or pharmaceutically acceptable salts, racemates, or enantiomers thereof, have a structure according to Formula I:

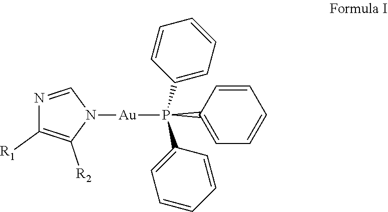

wherein R₁ and R₂ are independently selected from the group consisting of halo, cyano, hydroxyl, carboxyl, alkyl, aryl, amidyl, alkoxyl, and —C(O)—OR₃, wherein R₃ is a C1-C12 alkyl. In more specific embodiment, R₁ and R₂ are independently selected from the group consisting of halo, cyano, alkyl, and alkoxyl. In a more specific embodiment, R₁ and R₂ are independently selected from the group consisting of halo and cyano.

Suitable Formula I compounds for use in the methods disclosed herein include, but are not limited to, any of the compounds set forth in Table 1, alone or in combination.

TABLE 1
Formula I compounds

| Lab | | |
|---|---|---|
| Reference | | |
| Code | Structure | Name |
| DM20 | 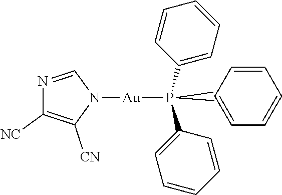 | 4,5-dicyano-imidazolate-1yl- gold(I)-triphenylphosphane |
| CS47 | 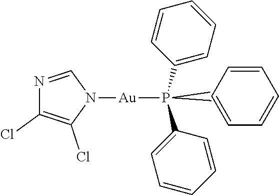 | 4,5-dichloro-imidazolate-1yl- gold(I)-triphenylphosphane |

In particular embodiments, the Formula I compound is selected from 4,5-dicyano-imidazolate-1yl-gold(I)-triphenylphosphane (DM20) and 4,5-dichloro-imidazolate-1yl-gold(I)-triphenylphosphane (CS47). Synthetic schemes for Formula I compounds, including DM20 and CS47, are available in Galassi, et al., Synthesis and characterization of azolate gold(I) phosphane complexes as thioredoxin reductase inhibiting antitumor agents, Dalton Transactions 41(17): 5307-18 (2012); and Galassi, et al., A study on the inhibition of dihydrofolate reductase (DHFR) from Escherichia coli by gold(i) phosphane compounds. X-ray crystal structures of (4,5-dichloro-1H-imidazolate-1-yl)-triphenylphosphane-gold(i) and (4,5-dicyano-1H-imidazolate-1-yl)-triphenylphosphane-gold(i), Dalton Transactions 44(7): 3043-56 (2015).

### Methods of Treating Lung Cancer and Inducing Ferroptosis

In one embodiment, a method of treating lung cancer in a subject in need thereof is provided, the method comprising administering to the subject an effective amount of an imidazolate gold compound according to Formula I, or a pharmaceutically acceptable salt, racemate, enantiomer, or derivative thereof. In another embodiment, a compound according to Formula I, or a pharmaceutically acceptable salt, racemate, enantiomer, or derivative thereof is provided, for use in treating lung cancer.

In particular embodiments, the Formula I compound is selected from 4,5-dicyano-imidazolate-1yl-gold(I)-triphenylphosphane (DM20) and 4,5-dichloro-imidazolate-1yl-gold(I)-triphenylphosphane (CS47).

In embodiments, the lung cancer is non-small cell lung cancer (NSCLC). In another embodiment, the lung cancer is KRAS-WT or KM lung cancer. In a specific embodiment, the lung cancer is KRAS-WT lung cancer.

In embodiments, the Formula I compound is administered orally, intravenously, intraperitoneally, intrathecally, intramuscularly, subcutaneously, rectally, intravaginally, sublingually, via inhalation, or transdermally. In a specific embodiment, the Formula I compound is administered intravenously, e.g., by injection or infusion.

In embodiments, the methods disclosed herein further comprise administering to the subject one or more additional anti-cancer agents. Suitable anti-cancer agents include one or more agents selected from a chemotherapeutic agent, an immunotherapeutic agent, and radiation therapy.

In a specific embodiment, the additional chemotherapeutic agent is selected from the group consisting of cisplatin, carboplatin, paclitaxel, albumin-bound paclitaxel, docetaxel, gemcitabine, vinorelbine, etopside, pemetrexed, and combinations thereof.

In another specific embodiment, the additional immunotherapeutic agent is selected from the group consisting of nivolumab, pembrolizumab, cemiplimab, atezolizumab, durvalumab, ipilimumab, tremelimumab, and combinations thereof.

In embodiments, the Formula I compound and the one or more additional anti-cancer agents are co-administered. “Co-administered,” as used herein, refers to administration of the Formula I compound and the additional anti-cancer agent such that both agents can simultaneously achieve a physiological effect, e.g., in a recipient subject. The two agents, however, need not be administered together. In certain embodiments, administration of one agent can precede administration of the other. Simultaneous physiological effect need not necessarily require presence of both agents in the circulation at the same time. However, in certain embodiments, co-administering results in both agents being simultaneously present in the subject. Thus, in embodiments, the Formula I compound and the additional anti-cancer agent may be administered concurrently or sequentially.

In another embodiment, a method of inducing ferroptosis in a lung cancer cell is provided, the method comprising contacting the lung cancer cell with an effective amount of a compound according to Formula I, or a pharmaceutically acceptable salt, racemate, enantiomer, or derivative thereof, according to embodiments of the present disclosure. In particular embodiments, the Formula I compound is selected from the group consisting of 4,5-dicyano-imidazolate-1yl-gold(I)-triphenylphosphane (DM20), 4,5-dichloro-imidazolate-1yl-gold(I)-triphenylphosphane (CS47), and combinations thereof.

In embodiments, the lung cancer is non-small cell lung cancer (NSCLC). In a specific embodiment, the lung cancer is KRAS-WT or KM lung cancer. In a more specific embodiment, the lung cancer is KRAS-WT lung cancer.

In embodiments, the method of inducing ferroptosis in a lung cancer cell is carried out in vitro or in vivo. Optionally, the method further comprises contacting the lung cancer cell with one or more additional anti-cancer agents. Suitable additional anti-cancer agents are selected from the group consisting of a chemotherapeutic agent, an immunotherapeutic agent, radiation therapy, and combinations thereof as disclosed herein.

### Pharmaceutical Compositions

In embodiments, a pharmaceutical composition is provided, the composition comprising a compound according to Formula I, or a pharmaceutically acceptable salt, racemate, enantiomer, or derivative thereof; and at least one pharmaceutically acceptable carrier. In embodiments, the pharmaceutical compositions disclosed herein are formulated for the treatment of lung cancer, i.e., for administration to a patient suffering from lung cancer.

The pharmaceutically acceptable excipient, or carrier, must be “acceptable” in the sense of being compatible with the other ingredients of the composition and not deleterious to the recipients thereof. The disclosure further includes a pharmaceutical composition, in combination with packaging material suitable for the pharmaceutical composition, including instructions for the use of the composition in the treatment of subjects in need thereof.

Pharmaceutical compositions include those suitable for oral, intravenous, intraperitoneal, intrathecal, intramuscular, subcutaneous, rectal, vaginal, sublingual, inhalation, or transdermal administration. In a specific embodiment, the pharmaceutical compositions are formulated for intravenous administration, e.g., by injection or infusion.

The compositions may be prepared by any methods well known in the art of pharmacy, for example, using methods such as those described in Remington: The Science and Practice of Pharmacy (21st ed., Lippincott Williams and Wilkins, 2005, see Part 5: Pharmaceutical Manufacturing). Suitable pharmaceutical carriers are well-known in the art. See, for example, Handbook of Pharmaceutical Excipients, Sixth Edition, edited by Raymond C. Rowe (2009). The skilled artisan will appreciate that certain carriers may be more desirable or suitable for certain modes of administration of an active ingredient. It is within the purview of the skilled artisan to select the appropriate carriers for a given vaccine composition.

For parenteral administration, suitable compositions include aqueous and non-aqueous sterile suspensions for intravenous administration. The compositions may be presented in unit dose or multi-dose containers, for example, sealed vials and ampoules.

As will be understood by those of skill in this art, the specific dose level for any particular subject will depend on a variety of factors, including the activity of the agent employed; the age, body weight, general health, and sex of the individual being treated; the time and route of administration; the rate of excretion; and the like.

In embodiments, an effective dose of a Formula I compound according to the present disclosure may range from about 0.01 mg/kg/day to about 100 mg/kg/day, or from about 0.01 mg/kg/day to about 10 mg/kg/day, or from about 0.1 mg/kg/day to about 100 mg/kg/day, or from about 0.1 mg/kg/day to about 10 mg/kg/day, or from about 1 mg/kg/day to about 10 mg/kg/day. In embodiments, the dose of a Formula 1 compound is at least about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 30, 40, 50, 60, 70, 80, 90, or 100 mg/kg/day, or any selected range of values therebetween.

### EXAMPLES

The following examples are given by way of illustration and are in no way intended to limit the scope of the present disclosure.

### Example 1. KRAS-WT Human Cancer Cell Lines are Dependent on the cyst(e)ine/TXNRD1/GSH/GPX4 Pathway

CRISPR-Cas9 knockout data from DepMap tool from BROAD institute (available at www.depmap.org). This screening assigns a CERES score, which defines whether a given human cancer cell line is dependent on a given gene. A gene with a CERES score of 0 or greater is considered non-essential, whereas a score lower than-0.5 indicates dependency, with-1 indicating all common essential genes.

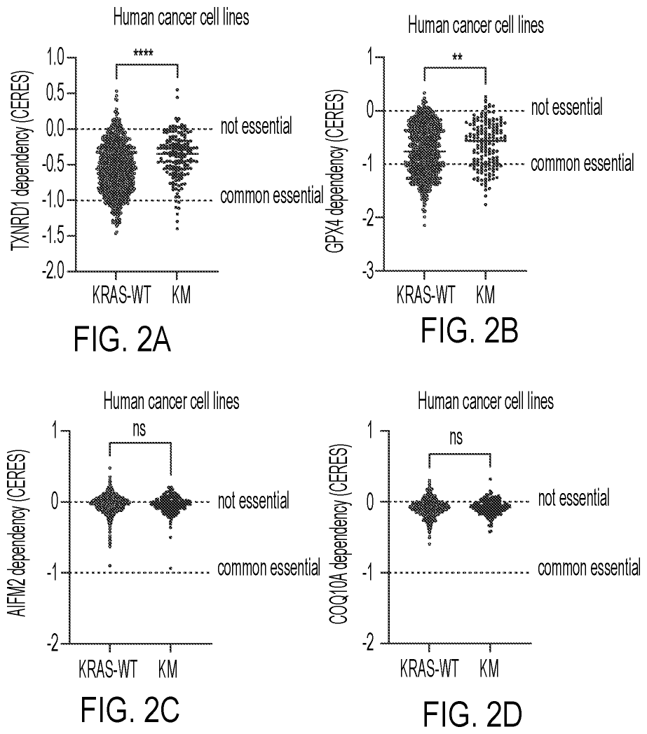

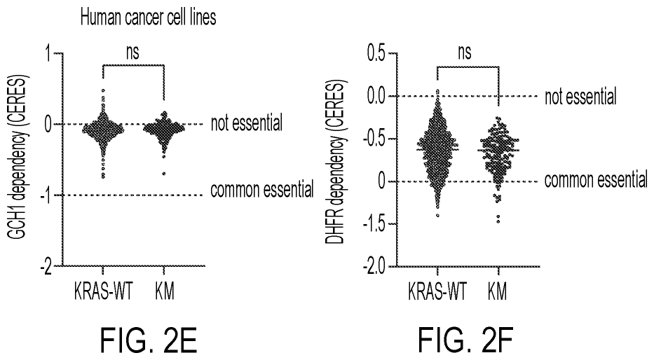

The three main anti-ferroptosis pathways (cyst(e)ine/TXNRD1/GSH/GPX4, CoQ10/FSP1, and GCH1/BH4/DHFR) were investigated to determine whether KM and KRAS-WT cancer cell lines have specific dependencies on genes involved in these processes. Results are set forth in FIG. 2A (TXNRD1), FIG. 2B (GPX4), FIG. 2C (AIFM2, also known as FSP1), FIG. 2D (COQ10A (also known as CoQ10), FIG. 2E (GCH1), and FIG. 2F (DHFR), which show the CERES scores from DepMap for the noted genes. Each dot represents a human cancer cell line. Statistics indicate Welch's t test with ns, p>0.05; **, p<0.01; and ****, p<0.0001.

Among the genes that were analyzed, it was found that KRAS-WT cancer cell lines are more dependent on thioredoxin reductase (gene TXNRD1, protein TrxR) and gluthathione peroxidase 4 (GPX4). These data suggest that KRAS-WT cancer cells use the cyst(e)ine/TXNRD1/GSH/GPX4 axis to escape ferroptosis.

### Example 2. KRAS-WT Human LC Cancer Cell Lines are Selectively More Sensitive to Gold Compounds than KMLC Cells and IMR₉₀ lung Fibroblasts

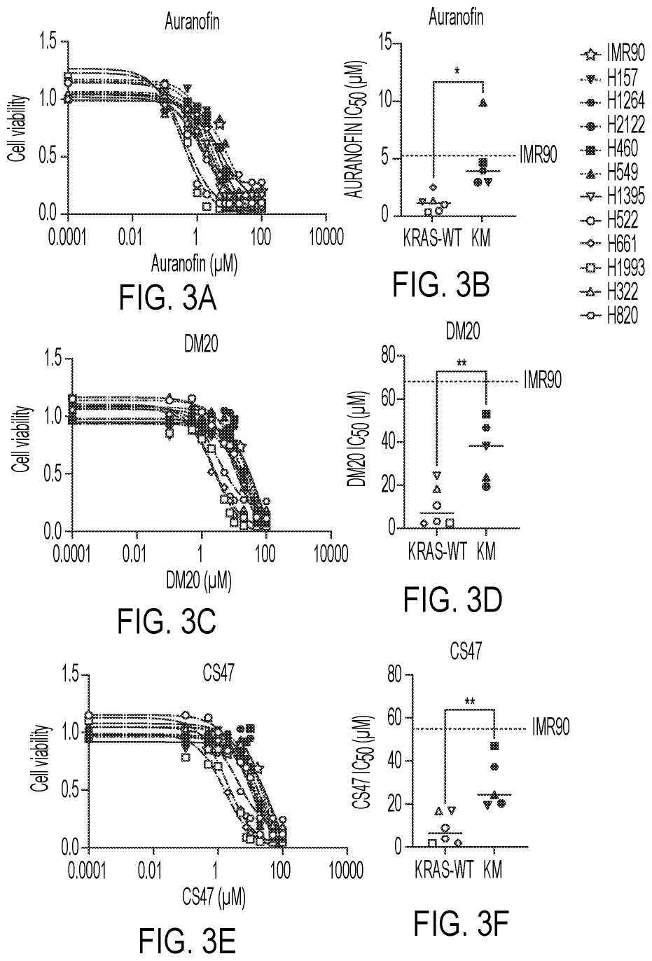

Experiments were carried out to determine whether DM20 and CS47 are reversible TrxR inhibitors capable of triggering ferroptosis in KRAS-WT lung cancer cells. DM20 and CS47 in concentrations ranging from 1 nM to 100 μM were tested on a panel of KM and KRAS-WT cell lines in MTT cell viability assays. The tested KM cell lines included H157, H1264, H2122, H460, and A549 cells. The tested KRAS-WT cell lines included H1395, H522, H661, H1993, H332, and H820. Auranofin was included as a positive drug control and the human lung cancer fibroblast cell line IMR₉₀ was included as a normal, non-transformed cell counterpart. MTT viability and IC₅₀ values were determined, results for which are shown in FIGS. 3A-3F, wherein each line or symbol corresponds to a human cancer cell line. Statistics indicate unpaired t test with *, p<0.05, and **, p<0.01.

Results show that KRAS-WT lung cancer cells are significantly and selectively more sensitive to DM20, CS47, and auranofin than KM lung cancer cells. Further, IMR₉₀ fibroblast control cells showed higher IC₅₀ values for all compounds, suggesting that the tested compounds are not indiscriminately toxic.

### Example 3. Ferrostatin-1, Cysteine, and NAC can Rescue the cell Viability of KRAS-WT Lung Cancer Cells Treated with DM20 and CS47

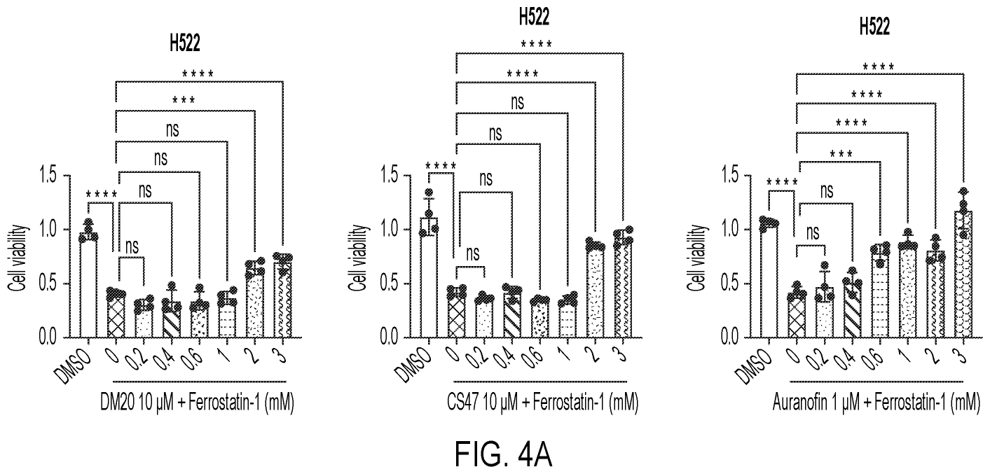

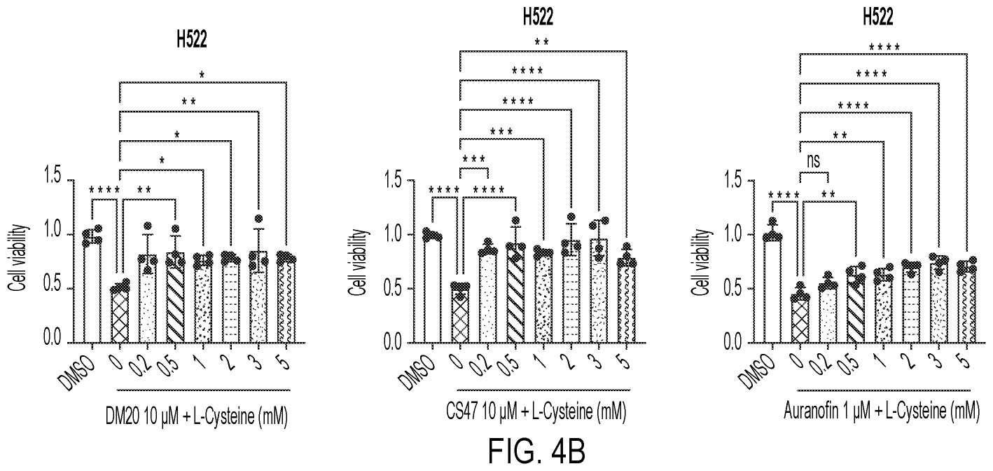

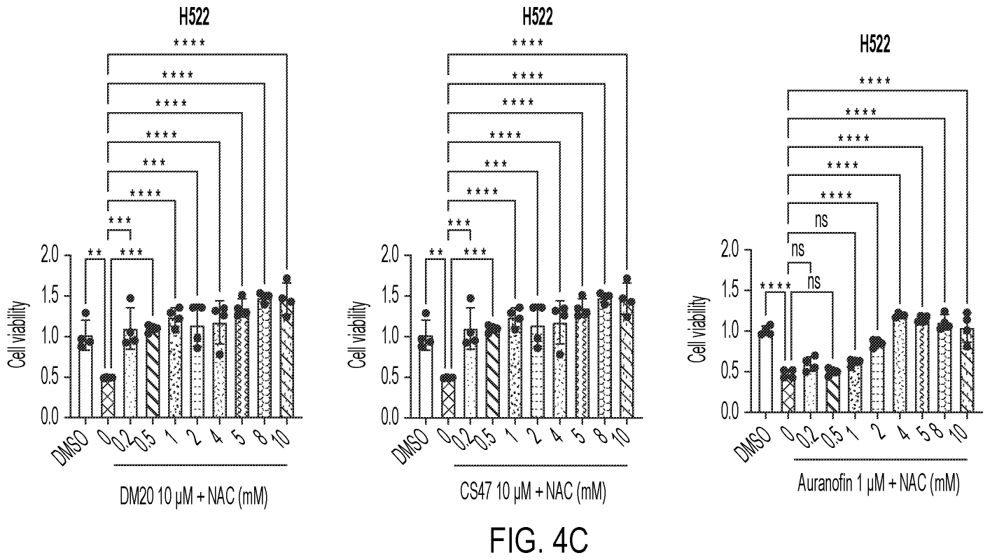

H522 KRAS-WT lung cancer cells were treated with DM20 (10 μM), CS47 (10 uM), or auranofin (1 μM) in combination with ferrostatin-1, L-cysteine, or n-acetyl-cysteine (all mM, 48 hours of treatment, in quadruplicate). Results are shown in FIGS. 4A-4C. Statistics indicate one-way ANOVA followed by Sidak's multiple comparisons analysis with ns, p>0.05; *, p<0.05: **, p<0.01; ***, p<0.001 and ****, p<0.0001.

Results show that the ferroptosis inhibitor ferrostatin-1 (Fer-1), the product of TXNRD1, cysteine, and its analogous n-acetyl-cysteine (NAC) can rescue the cell viability of KRAS-WT LC cells treated with DM20, CS47 and auranofin.

### Example 4. Treatment with DM20 and CS47 Depletes GPX4 and Induces Lipid Peroxidation in KRAS-WT Lung Cancer Cell Lines

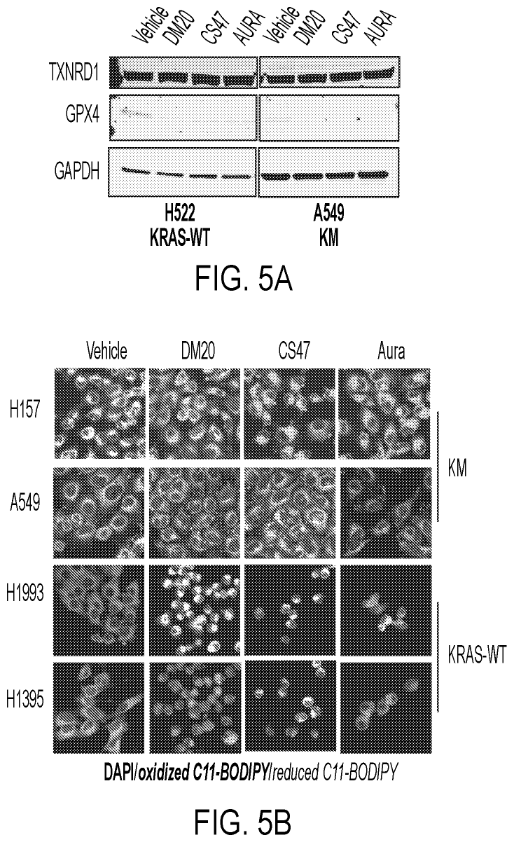

H522 KRAS-WT lung cancer cells and A546 KM lung cancer cells were treated with vehicle (negative control), DM20 (10 μM), CS47 (10 μM), and auranofin (1 μM, positive control) for 48 hours. Protein lysates were extracted from the cells and Western blot analysis was carried out to detect expression of TXNRD1 and GPX4 in the cell lines after treatment, with GAPDH expression detected as a control. Results are shown in FIG. 5A and indicate that treatment with DM20 and CS47 depletes GPX4 expression in the tested cell lines.

H157 and A549 KM lung cancer cells and H193 and H1395 KRAS-WT lung cancer cells were treated with vehicle (negative control), DM20 (10 μM), CS47 (10 μM), or auranofin (1 μM positive control) for 48 hours. Cells were then stained with DAP, oxidized C11-BODIPY, and reduced C11-BODIPY. C11-BODIPY is a ferroptosis lipid sensor that switches from red to green fluorescence upon oxidation.

As shown in FIG. 5B, results show that treatment with DM20 and CS47 induces lipid peroxidation, but only in KRAS-WT cells. The data is consistent with the CRISPR data depicted in FIG. 2, showing that GPX4 and TXNRDI are specific vulnerabilities of KRAS-WT cancer cells.

Taken together, these data indicate that DM20 and CS47 target the cyst(e)ine/TXNRD1/GSH/GPX4 axis and induce ferroptosis in KRAS-WT lung cancer cells.

Aspects of the present disclosure can be described with reference to the following numbered clauses, with preferred features laid out in dependent clauses.
1. A method of treating lung cancer in a subject in need thereof, the method comprising administering to the subject an effective amount of an imidazolate gold compound according to Formula I, or a pharmaceutically acceptable salt, racemate, or enantiomer thereof:

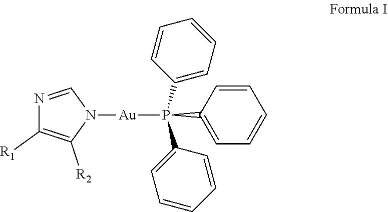

wherein R₁ and R₂ are independently selected from the group consisting of halo, cyano, hydroxyl, carboxyl, alkyl, aryl, amidyl, alkoxyl, and —C(O)—OR₃, wherein R₃ is a C1-C12 alkyl.
2. The method according to clause 1, wherein the lung cancer is KRAS-wild type lung cancer.
3. The method according to any of the preceding clauses, wherein the imidazolate gold compound is selected from the group consisting of 4,5-dicyano-imidazolate-1yl-gold(I)-triphenylphosphane (DM20), 4,5-dichloro-imidazolate-1yl-gold(I)-triphenylphosphane (CS47), and combinations thereof.
4. The method according to any of the preceding clauses, wherein the imidazolate gold compound is administered by injection or infusion.
5. The method according to any of the preceding clauses, further comprising administering to the subject one or more additional anti-cancer agents.
6. The method according to clause 5, wherein the one or more additional anti-cancer agents is selected from the group consisting of a chemotherapeutic agent, an immunotherapeutic agent, and radiation therapy.
7. The method according to clause 6, wherein the chemotherapeutic agent is selected from the group consisting of cisplatin, carboplatin, paclitaxel, albumin-bound paclitaxel, docetaxel, gemcitabine, vinorelbine, etopside, pemetrexed, and combinations thereof.
8. The method according to clause 6, wherein the immunotherapeutic agent is selected from the group consisting of nivolumab, pembrolizumab, cemiplimab, atezolizumab, durvalumab, ipilimumab, tremelimumab, and combinations thereof.
9. The method according to any of the preceding clauses, wherein the subject is a human.
10. A method of inducing ferroptosis in a lung cancer cell, the method comprising contacting the lung cancer cell with an effective amount of a compound according to Formula I, or a pharmaceutically acceptable salt, racemate, or enantiomer thereof.
11. The method according to clause 10, wherein the lung cancer cell is a KRAS-wild type lung cancer cell.
12. The method according to any of clauses 10-11, wherein the imidazolate gold compound is selected from the group consisting of 4,5-dicyano-imidazolate-1yl-gold(I)-triphenylphosphane (DM20), 4,5-dichloro-imidazolate-1 yl-gold(I)-triphenylphosphane (CS47), and combinations thereof.
13. The method according to any of clauses 10-12, wherein the method is carried out in vitro or in vivo.
14. The method according to any of clauses 10-13, further comprising contacting the lung cancer cell with one or more additional anti-cancer agents.
15. The method according to clause 14, wherein the one or more additional anti-cancer agents is selected from the group consisting of a chemotherapeutic agent, an immunotherapeutic agent, and radiation therapy.
16. The method according to clause 15, wherein the chemotherapeutic agent is selected from the group consisting of cisplatin, carboplatin, paclitaxel, albumin-bound paclitaxel, docetaxel, gemcitabine, vinorelbine, etopside, pemetrexed, and combinations thereof.
17. The method according to clause 15, wherein the immunotherapeutic agent is selected from the group consisting of nivolumab, pembrolizumab, cemiplimab, atezolizumab, durvalumab, ipilimumab, tremelimumab, and combinations thereof.
18. A compound according to Formula I, or a pharmaceutically acceptable salt, racemate, or enantiomer thereof for use in treating lung cancer.
19. The compound for use according to clause 18, wherein the lung cancer is KRAS-wild type lung cancer.
20. The compound for use according to any of clauses 18-19, wherein the imidazolate gold compound is selected from the group consisting of 4,5-dicyano-imidazolate-1yl-gold(I)-triphenylphosphane (DM20), 4,5-dichloro-imidazolate-1yl-gold(I)-triphenylphosphane (CS47), and combinations thereof.
21. The compound for use according to any of clauses 18-20, wherein treating comprises administering the imidazolate gold compound to a subject by injection or infusion.
22. The compound for use according to any of clauses 18-21, further comprising administering to the subject one or more additional anti-cancer agents.
23. The compound for use according to clause 22, wherein the one or more additional anti-cancer agents is selected from the group consisting of a chemotherapeutic agent, an immunotherapeutic agent, and radiation therapy.
24. The compound for use according to clause 23, wherein the chemotherapeutic agent is selected from the group consisting of cisplatin, carboplatin, paclitaxel, albumin-bound paclitaxel, docetaxel, gemcitabine, vinorelbine, etopside, pemetrexed, and combinations thereof.
25. The compound for use according to clause 23, wherein the immunotherapeutic agent is selected from the group consisting of nivolumab, pembrolizumab, cemiplimab, atezolizumab, durvalumab, ipilimumab, tremelimumab, and combinations thereof.
26. The compound for use according to any of clauses 18-25, wherein the subject is a human.
27. A pharmaceutical composition for use in treating lung cancer, the composition comprising: a compound according to Formula I, or a pharmaceutically acceptable salt, racemate, or enantiomer thereof; and at least one pharmaceutically acceptable excipient.

All documents cited are incorporated herein by reference; the citation of any document is not to be construed as an admission that it is prior art with respect to the present invention.

The foregoing description is illustrative of particular embodiments of the invention but is not meant to be a limitation upon the practice thereof. While particular embodiments have been illustrated and described, it would be obvious to one skilled in the art that various other changes and modifications can be made without departing from the spirit and scope of the invention. It is therefore intended to cover in the appended claims all such changes and modifications that are within the scope of this invention.

1. A method of treating lung cancer in a subject in need thereof, the method comprising administering to the subject an effective amount of an imidazolate gold compound according to Formula I, or a pharmaceutically acceptable salt, racemate, or enantiomer thereof: 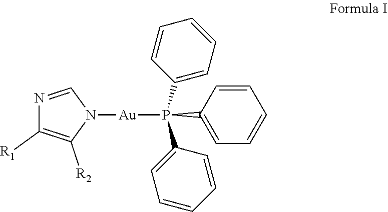
wherein R₁ and R₂ are independently selected from the group consisting of halo, cyano, hydroxyl, carboxyl, alkyl, aryl, amidyl, alkoxyl, and —C(O)—OR₃, wherein R₃ is a C1-C12 alkyl.

2. The method according to claim 1, wherein the lung cancer is KRAS-wild type lung cancer.

3. The method according to claim 1, wherein the imidazolate gold compound is selected from the group consisting of 4,5-dicyano-imidazolate-1yl-gold(I)-triphenylphosphane (DM20), 4,5-dichloro-imidazolate-1yl-gold(I)-triphenylphosphane (CS47), and combinations thereof.

4. The method according to claim 1, wherein the imidazolate gold compound is administered by injection or infusion.

5. The method according to claim 1, further comprising administering to the subject one or more additional anti-cancer agents.

6. The method according to claim 5, wherein the one or more additional anti-cancer agents is selected from the group consisting of a chemotherapeutic agent, an immunotherapeutic agent, and radiation therapy.

7. The method according to claim 6, wherein the chemotherapeutic agent is selected from the group consisting of cisplatin, carboplatin, paclitaxel, albumin-bound paclitaxel, docetaxel, gemcitabine, vinorelbine, etopside, pemetrexed, and combinations thereof.

8. The method according to claim 6, wherein the immunotherapeutic agent is selected from the group consisting of nivolumab, pembrolizumab, cemiplimab, atezolizumab, durvalumab, ipilimumab, tremelimumab, and combinations thereof.

9. The method according to claim 1, wherein the subject is a human.

10. A method of inducing ferroptosis in a lung cancer cell, the method comprising contacting the lung cancer cell with an effective amount of a compound according to Formula I, or a pharmaceutically acceptable salt, racemate, or enantiomer thereof: 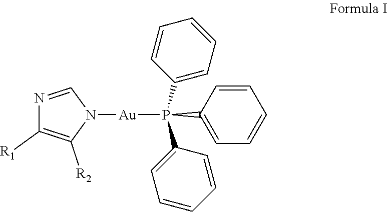
wherein R₁ and R₂ are independently selected from the group consisting of halo, cyano, hydroxyl, carboxyl, alkyl, aryl, amidyl, alkoxyl, and —C(O)—OR₃, wherein R₃ is a C1-C12 alkyl.

11. The method according to claim 10, wherein the lung cancer cell is a KRAS-wild type lung cancer cell.

12. The method according to claim 10, wherein the imidazolate gold compound is selected from the group consisting of 4,5-dicyano-imidazolate-1yl-gold(I)-triphenylphosphane (DM20), 4,5-dichloro-imidazolate-1yl-gold(I)-triphenylphosphane (CS47), and combinations thereof.

13. The method according to claim 10, wherein the method is carried out in vitro or in vivo.

14. The method according to claim 10, further comprising contacting the lung cancer cell with one or more additional anti-cancer agents.

15. The method according to claim 14, wherein the one or more additional anti-cancer agents is selected from the group consisting of a chemotherapeutic agent, an immunotherapeutic agent, and radiation therapy.

16. The method according to claim 15, wherein the chemotherapeutic agent is selected from the group consisting of cisplatin, carboplatin, paclitaxel, albumin-bound paclitaxel, docetaxel, gemcitabine, vinorelbine, etopside, pemetrexed, and combinations thereof.

17. The method according to claim 15, wherein the immunotherapeutic agent is selected from the group consisting of nivolumab, pembrolizumab, cemiplimab, atezolizumab, durvalumab, ipilimumab, tremelimumab, and combinations thereof.

18-26. (canceled)

27. A pharmaceutical composition for use in treating lung cancer, the composition comprising:
a compound according to Formula I, or a pharmaceutically acceptable salt, racemate, or enantiomer thereof: 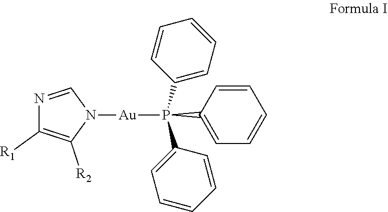
wherein R₁ and R₂ are independently selected from the group consisting of halo, cyano, hydroxyl, carboxyl, alkyl, aryl, amidyl, alkoxyl, and —C(O)—OR₃, wherein R₃ is a C1-C12 alkyl; and
at least one pharmaceutically acceptable excipient.

28. The pharmaceutical composition according to claim 27, wherein the compound is selected from the group consisting of 4,5-dicyano-imidazolate-lyl-gold(I)-triphenylphosphane (DM20), 4,5-dichloro-imidazolate-1yl-gold(I)-triphenylphosphane (CS47), and combinations thereof.

29. The pharmaceutical composition according to claim 27, wherein the pharmaceutical composition is formulated for injection or infusion.
